(19) United States
(12) Patent Application Publication
(10) Pub. No.: US 2026/0256572 A1
(43) Pub. Date: Sep. 3, 2026

(54) SUTURELESS REPLACEMENT VALVE HOLDER

(71) Applicant: BOSTON SCIENTIFIC SCIMED, INC., MAPLE GROVE, MN (US)

(72) Inventors: Donal Kyne, St. Louis Park, MN (US); Jessica D. Morlock, Hopkins, MN (US); Sean Shanley, Galway (IE); Kristi Mae Flury, Maple Grove, MN (US); Leonardo Orlando Pagotti Longo, Sao Paulo (BR); Chris Dyke, Galway (IE); Cathy Woods, Galway (IE); Celia Faria, Galway (IE); Daciella Ferreira Araujo Noguiera, Sabara (BR)

(73) Assignee: BOSTON SCIENTIFIC SCIMED, INC., MAPLE GROVE, MN (US)

(21) Appl. No.: 19/541,839

(22) Filed: Feb. 17, 2026

Related U.S. Application Data
(60) Provisional application No. 63/759,698, filed on Feb. 18, 2025.

(51) Int. Cl. A61F 2/24

(52) U.S. Cl. CPC A61F 2/2409; A61F 2220/0025; A61F 2230/0006

(57) ABSTRACT

A prosthetic heart valve holder includes a ring body defining a central opening, and a plurality of mating features disposed on the ring body and facing the central opening. Each mating feature may be configured to engage with one of a plurality of stabilization arches of a prosthetic heart valve when the prosthetic heart valve is in an expanded configuration.

### CROSS-REFERENCE TO RELATED APPLICATIONS

This application claims the benefit of priority of U.S. Provisional Application No. 63/759,698 filed February 18, 2025, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to holder for a replacement heart valve implant.

### BACKGROUND

A wide variety of intracorporeal medical devices have been developed for medical use, and more specifically for intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example prosthetic heart valve holder includes a ring body defining a central opening, and a plurality of mating features disposed on the ring body and facing the central opening, each mating feature configured to engage with one of a plurality of stabilization arches of a prosthetic heart valve when the prosthetic heart valve is in an expanded configuration.

Alternatively or additionally to the embodiment above, each mating feature is configured to engage one of the plurality of stabilization arches at at least two contact points.

Alternatively or additionally to any of the embodiments above, the plurality of mating features includes three mating features positioned at 120-degree intervals.

Alternatively or additionally to any of the embodiments above, each of the plurality of mating features includes a tab extending axially upwards from a top surface of the ring body, and at least a first tab includes a post extending from the tab radially into the central opening.

Alternatively or additionally to any of the embodiments above, each of the plurality of mating features includes a cut-out portion of the ring body adjacent a base of the tab, with the post centered above the cut-out portion.

Alternatively or additionally to any of the embodiments above, each of the tabs include the post extending radially into the central opening.

Alternatively or additionally to any of the embodiments above, a second tab and a third tab each includes an opening extending radially through the tab, wherein the prosthetic heart valve holder further includes a rod configured to extend across a portion of the central opening and through the openings in the second and third tabs.

Alternatively or additionally to any of the embodiments above, the ring body includes a handle, wherein at least a portion of the handle is configured to extend axially upwards above an upper surface of the ring body and extending across the central opening.

Alternatively or additionally to any of the embodiments above, the handle has a first configuration in which it extends laterally across the central opening and does not extend axially above the upper surface of the ring body, and a second configuration in which at least the portion of the handle rotates to extend axially above the upper surface of the ring body.

Alternatively or additionally to any of the embodiments above, each of the plurality of mating features includes an arm having a first portion extending axially upwards from an upper surface of the ring body, and a second portion extending at an angle from the first portion.

Another example prosthetic heart valve holder includes a ring body, and a plurality of engagement members extending axially upward from an upper surface of the ring body, each of the plurality of engagement members configured to engage an apex of one of a plurality of stabilization arches of a prosthetic heart valve when the prosthetic heart valve is in an expanded configuration.

Alternatively or additionally to the embodiment above, each of the plurality of engagement members includes a notch in the ring body, each notch configured to engage opposing sides of a single stabilization arch.

Alternatively or additionally to any of the embodiments above, the plurality of engagement members each includes a tab extending axially from the ring body, wherein at least one tab has an extension configured to engage the apex of one of the plurality of stabilization arches at a position axially spaced apart from the notch.

Alternatively or additionally to any of the embodiments above, each of the plurality of engagement members includes a tab extending axially upwards from a top surface of the ring body, and at least a first tab includes a post extending from the tab radially into a central opening of the ring body.

Alternatively or additionally to any of the embodiments above, each of the tabs include the post extending from the tab radially into the central opening.

Alternatively or additionally to any of the embodiments above, a second tab and a third tab each includes an opening extending radially through the tab, wherein the prosthetic heart valve holder further includes a rod configured to extend across a portion of the central opening and through the openings in the second and third tabs, wherein the apex of each stabilization arch extends over the rod.

Alternatively or additionally to any of the embodiments above, the ring body includes a handle, wherein at least a portion of the handle is configured to extend axially upwards above the upper surface of the ring body and to extend across a central opening of the ring body.

An example method of securing an expandable prosthetic heart valve to a valve holder includes compressing commissure posts of a prosthetic heart valve in an expanded configuration, wherein each commissure post joins adjacent stabilization arches, aligning an apex of each one of a plurality of stabilization arches of the prosthetic heart valve with a corresponding mating feature on the valve holder, and allowing the prosthetic heart valve to expand such that each stabilization arch engages with one mating feature.

Alternatively or additionally to the embodiment above, the valve holder includes a ring body defining a central opening and the mating feature includes a plurality of tabs extending axially upward from an upper surface of the ring body, wherein at least a first tab includes a post extending radially into the central opening, wherein aligning the apex of each stabilization arch includes inserting the prosthetic heart valve into the central opening from a lower surface of the ring body and placing the apex of one stabilization arch over the post.

Alternatively or additionally to any of the embodiments above, the ring body includes three tabs each with the post extending radially into the central opening, wherein the ring body further includes a notch aligned below each post, wherein aligning the apex of each stabilization arch includes inserting the prosthetic heart valve into the central opening from the lower surface of the ring body and placing the apex of each stabilization arch over one of the posts and opposing sides of each stabilization arch within the notch.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1A illustrates an example prosthetic valve holder with an attached prosthetic heart valve;

FIG. 1B is a perspective view of the prosthetic valve holder of FIG. 1A with a portion of the prosthetic heart valve shown;

FIG. 2 is a perspective view of another example prosthetic valve holder;

FIGS. 3A and 3B are perspective views of a further example prosthetic valve holder with a handle in the flat and folded configurations, respectively;

FIGS. 4A and 4B are perspective view of additional example prosthetic valve holders; and

FIGS. 5A-5C illustrate a method of loading and unloading a prosthetic heart valve onto the prosthetic valve holder of FIG. 1.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve could lead to a serious health condition and/or death if not dealt with properly. Treatment of defective heart valves often requires the repair or outright replacement of the defective heart valve. Disclosed herein is an apparatus, system, and/or method that may be used in a portion of the cardiovascular system in order to treat, and/or repair a native heart valve (e.g., aortic, etc.). In some cases, a replacement heart valve implant may be delivered percutaneously via a delivery catheter and thus may be much less invasive to the patient than surgical approaches.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the frame strut”, or other features may be equally referred to all instances and quantities beyond one of said feature unless clearly stated to the contrary. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient’s anatomy, such as but not limited to, arteries, veins, and/or other body lumens.

Prosthetic heart valves may be used to replace any type of heart valve (e.g., a mitral valve, an aortic valve, etc.). The prosthetic heart valve may be implanted (e.g., surgically or through transcatheter delivery) in a mammalian heart. Many prosthetic heart valves include an expandable stent structure with valve leaflets attached to the inner surface of the stent structure. The expandable stent structure may be configured to shift from a collapsed configuration to an expanded configuration. In some embodiments, the expandable stent structure may be self-expanding, and may be biased toward the expanded configuration.

Prosthetic heart valves are conventionally sutured to a valve holder and packaged in glutaraldehyde in their expanded configuration for storage and transport. The suture is then cut during valve preparation in a catheter lab for loading the prosthetic heart valve onto a delivery device. Suturing is a time-consuming process generally requiring suturing of three knots and inspection before packaging. The valve must be secured in the package (i.e., bottle) due to the potential risk of damage during transit. The valve holder functions to support the stent portion of the prosthetic heart valve and additionally may include serial numbers or other identifying markings that aid in the logistics and confirming authenticity of the attached valve.

Once the prosthetic heart valve is removed from the bottle during preparation in the catheter lab, a user (e.g., practitioner, clinician, etc.) must rinse the heart valve to remove glutaraldehyde before carefully cutting away the securement sutures with a blade. The use of a blade to cut the sutures holding the valve to the holder carries a risk of damage to the stent and valve leaflet portions of the prosthetic heart valve. A sutureless alternative valve holder will provide the advantages of faster processing on the production line, faster preparation in the catheter lab, and removing the requirement of using a blade and risking damage to the valve and stent.

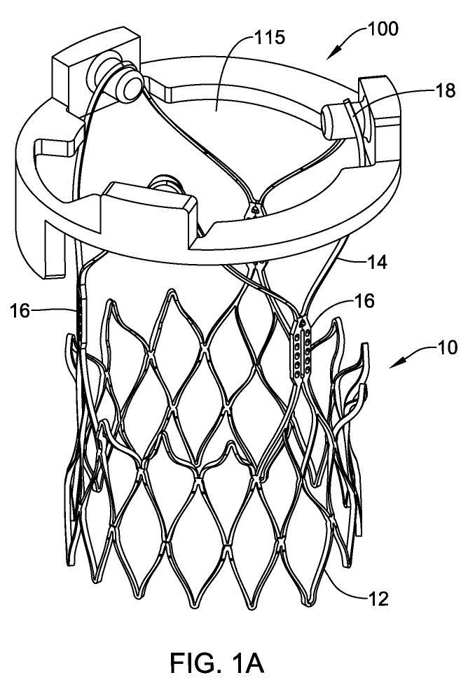

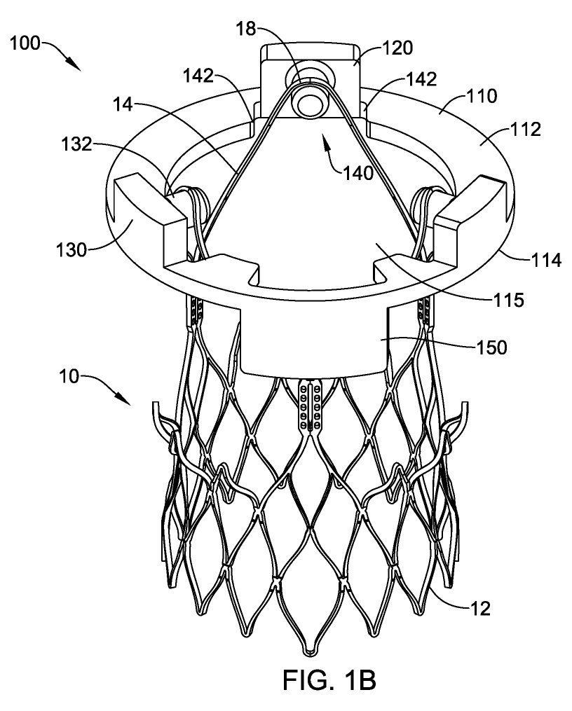

FIG. 1A illustrates a prosthetic heart valve 10 coupled to an example prosthetic valve holder 100. The prosthetic heart valve 10 may include a plurality of struts forming an expandable framework 12. The expandable framework 12 may be made of nitinol and include a plurality of stabilization arches 14 and a plurality of commissures 16 between adjacent stabilization arches 14. The prosthetic heart valve 10 is movable between a compressed delivery configuration and an expanded configuration. The prosthetic heart valve 10 may be coupled to the valve holder 100 in the expanded configuration. Example prosthetic heart valves 10 include ACURATE Prime™ and ACURATE neo2™ available from Boston Scientific of Marlborough, MA, USA. Each stabilization arch 14 has an apex 18. The valve leaflets are not shown in FIG. 1A. The prosthetic heart valve 10 is shown extending at least partially within a central opening 115 of a valve holder 100, with the apex 18 of each stabilization arch 14 coupled to the valve holder 100. The valve holder 100 allows the prosthetic heart valve 10 to be stored for transport and rinsed prior to use without excessive handling of the valve, and without the need for suturing to secure the prosthetic heart valve 10 to the valve holder 100.

FIG. 1B illustrates the valve holder 100 in detail. The valve holder 100 may include a ring body 110 having an upper surface 112 and a lower surface 114 and defining a central opening 115. The ring body 110 may be molded from a polymer. A plurality of engagement members or mating features 120 may be disposed on the ring body 110 facing the central opening 115. Each mating feature 120 may be configured to engage with one of the plurality of stabilization arches 14 of the prosthetic heart valve 10 when the prosthetic heart valve is in the expanded configuration, as shown in FIG. 1A. In the embodiment shown in FIG. 1B, three mating features 120 are positioned on the ring body 110 at 120-degree intervals. In other embodiments, only two mating features 120 may be provided, or more than three. In general, one mating feature 120 may be provided for each stabilization arch 14 on the prosthetic heart valve 10.

Each of the plurality of mating features 120 may include a tab 130 extending perpendicularly (i.e. axially upwards) from the upper surface 112 of the ring body 110, as shown in FIG. 1B. At least a first tab 130 includes an extension or post 132 extending from the tab radially into the central opening 115. In the illustrated embodiment, each of the tabs 130 have a post 132 extending radially into the central opening 115. The mating features 120 in this embodiment also include a notch or cut-out portion 140 of the ring body 110 adjacent a base of the tab 130, with the post 132 centered above the cut-out portion 140. This mating feature structure allows each stabilization arch 14 to engage one mating feature 120 at at least two contact points. As shown in FIG. 1B, a stabilization arch 14 of the prosthetic valve engages the mating feature 120 at a contact point. The other stabilization arches 14 would engage the two other mating features 120 at their respective contact points. Opposing sides of a single stabilization arch 14 may contact each of the opposing edges 142 of the cut-out portion 140 and the apex 18 engages the top of the post 132. These three contact points may securely hold the stabilization arches 14 and prevent axial and rotational movement of the prosthetic heart valve 10 relative to the valve holder 100. In some embodiments, the ring body 110 may include a panel 150 extending axially therefrom, which may include size information and/or a serial number for the prosthetic heart valve.

The inner diameter of the ring body 110 within the central opening 115 may be less than the outer diameter of the prosthetic heart valve in the region of the stabilization arches 14. This allows the prosthetic heart valve to be slightly compressed when coupled to the valve holder 100, providing an additional level of securement. Prosthetic heart valves 10 may have an outer extent (diameter) of about 21 millimeters (mm), about 23 mm, about 25 mm, about 27 mm, about 30 mm, etc. in an unconstrained configuration (e.g., in the expanded configuration). In order to accommodate the various sizes of prosthetic heart valves 10, the inner diameter of the central opening 115 of the valve holder 100 may be between 19 mm (0.75 inch) and 27 mm (1.06 inch). In some embodiments, the inner diameter of the central opening 115 may be 1 to 3 mm (0.04 to 0.12 inch) less than the outer diameter of the prosthetic heart valve. As the prosthetic heart valve 10 is in its expanded configuration, the stabilization arches 14 may be somewhat compressed within the valve holder, which further secures the prosthetic heart valve to the valve holder 100.

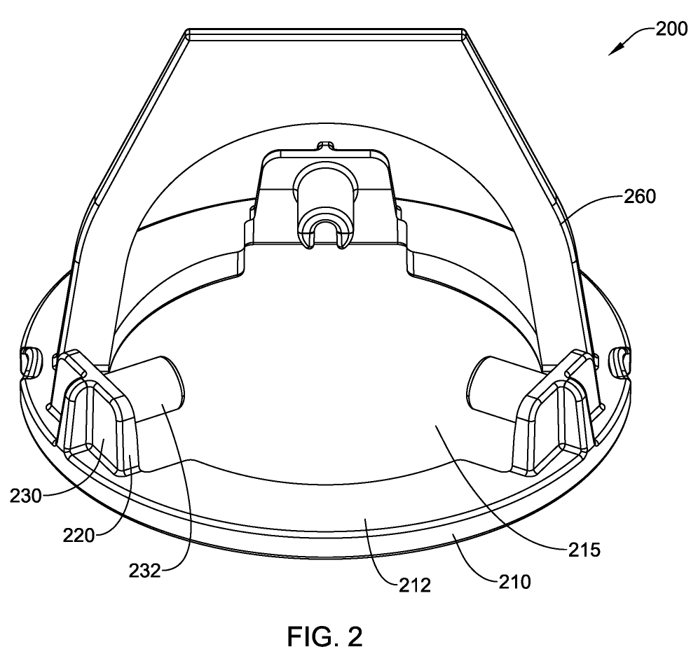

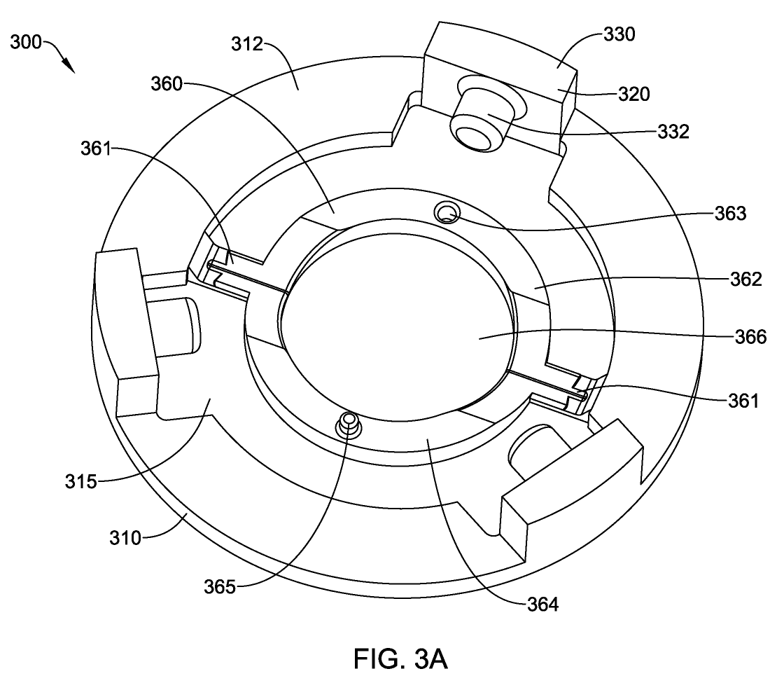

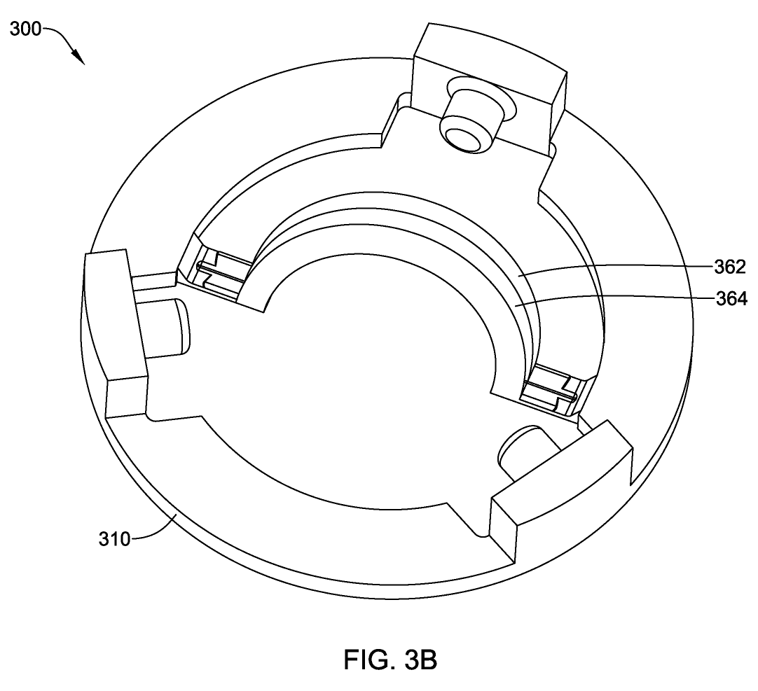

FIGS. 2, 3A, and 3B illustrate prosthetic valve holders 200, 300 with different supportive arches or handles 260, 360. Each prosthetic valve holder 200, 300 includes a ring body 210, 310 defining a central opening 215, 315 and having a plurality of mating features 220, 320 disposed on the ring body 210, 310 facing the central opening 215, 315. Similar to the valve holder 100 described above, the mating features 220, 320 may each include a tab 230, 330 extending perpendicularly (i.e., axially upwards) from the upper surface 212, 312 of the ring body 210, 310 with a post 232, 332 extending from the tab radially into the central opening 215, 315. The only difference between the valve holders 200, 300 in FIGS. 2, 3A, and 3B and the valve holder 100 in FIG. 1B may be the handles 260, 360.

The handle 260 in FIG. 2 may extend axially upwards above the upper surface 212 of the ring body 210 and extend across the central opening 215. In some embodiments, a serial number or size indicator may be printed or engraved on the handle 260. The handle 360 in FIGS. 3A and 3B may have a first, flat configuration (shown in FIG. 3A) in which the handle 360 extends laterally across the central opening 315 and does not extend axially above the upper surface 312 of the ring body 310. The handle 360 in FIGS. 3A and 3B may have a second, folded configuration (shown in FIG. 3B) in which at least a portion of the handle 360 rotates to extend axially above the upper surface 312 of the ring body 310. As illustrated, the handle 360 includes connectors 361 extending radially inward from the ring body 310, and a first handle portion 362 and second handle portion 364 each rotatably coupled to the connectors 361. In some embodiments, the first and second handle portions 362, 364 may be semi-circular. The first and second handle portions 362, 364 are configured to rotate or fold upwards and into contact with each other, forming a folded handle extending above the upper surface 312 of the ring body 310 and extending across the central opening 315. See FIG. 3B. In some embodiments, the first handle portion 362 may include a recess 363 and the second handle portion 364 may include a protrusion 365 configured to be inserted into the recess 363 in a snap fit or friction fit when the first and second handle portions 362, 364 are pushed together to secure the first and second handle portions 362, 364 to one another. The handles 260, 360 provide a location for the user to grasp the valve holder 200, 300, allowing the heart valve to be loaded into a storage container and removed from the container and rinsed while minimizing contact with the heart valve.

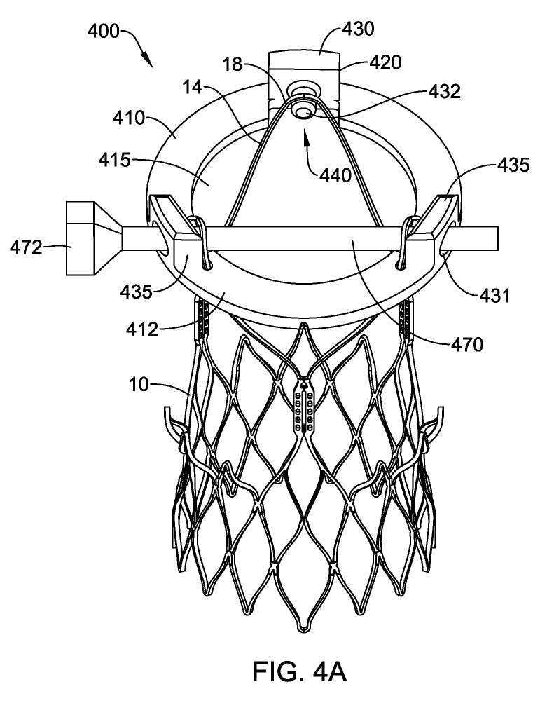

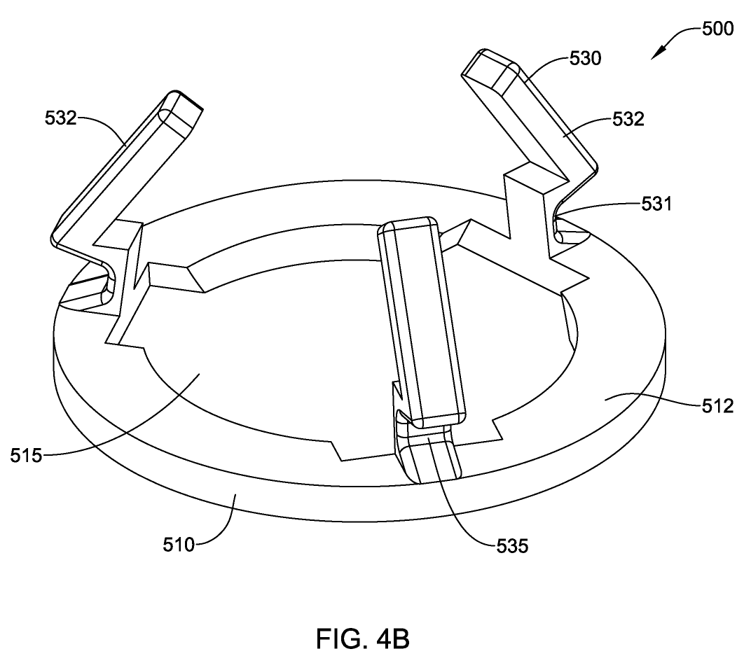

FIG. 4A illustrates an embodiment of a two-piece prosthetic heart valve holder 400 that includes a ring body 410 having an upper surface 412 and defining a central opening 415. A plurality of mating features 420 may be disposed on the ring body 410 facing the central opening 415. A first mating feature 420 may include a tab 430 extending perpendicularly (i.e., axially upwards) from the upper surface 412 of the ring body 410. The first tab 430 includes a post 432 extending from the tab radially into the central opening 415. Second and third tabs 435 also extend perpendicularly (i.e., axially upwards) from the upper surface 412 of the ring body 410 and each has an opening 431 extending radially therethrough. The prosthetic heart valve holder 400 further includes a rod 470 configured to extend through the openings 431 in the second and third tabs 435 and across a portion of the central opening 415, as shown in FIG. 4A. The rod 470 may have an enlarged end 472 to prevent it from being pushed completely through the openings 431 in the second and third tabs 435. In other embodiments, the rod 470 may be tapered along all or a portion of its length with a larger end adjacent the enlarged end 472 so as the rod is pushed through the openings in the second and third tabs 435, an increasingly tighter friction fit is achieved. In some embodiments a tapered rod without the enlarged end 472 may be provided, with just the taper securing the rod within the openings 431 in the second and third tabs 435.

The ring body 410 may include a notch or cut-out portion 440 adjacent a base of each of the first, second, and third tabs 430, 435, with the post 432 or opening 431 centered above the cut-out portion 440. The prosthetic heart valve may be secured to the valve holder 400 by placing the apex 18 of a first stabilization arch 14 over the post 432 of the first tab 430. The second and third stabilization arches may be aligned with the openings 431 in the second and third tabs 435 and the rod may be advanced through the openings 431 and under the apex 18 of each stabilization arch 14. See FIG. 4A. The stabilization arch 14 may also contact each of the opposing edges of the cut-out portion 440. These three contact points may securely hold the stabilization arches 14 and prevent axial and rotational movement of the prosthetic heart valve 10 relative to the valve holder 400.

FIG. 4B illustrates another embodiment of a prosthetic heart valve holder 500 that includes a ring body 510 having an upper surface 512 and defining a central opening 515. A plurality of mating features 530 may be disposed on the ring body 510 facing the central opening 515. Each mating feature 530 may include an arm having a first portion 531 extending axially upwards from the upper surface 512 of the ring body and a second portion 532 extending at an angle from the first portion 531. The first portion 531 of each mating feature 530 may include a groove or indentation 535 on a radially outward surface thereof, configured to receive the apex of a stabilization arch of the prosthetic heart valve. The prosthetic heart valve is inserted into the central opening 515 from the bottom of the ring body 510 as it is oriented in FIG. 4B. Each stabilization arch is moved over one of the mating features 530 and positioned within the indentation 535 to secure the prosthetic heart valve to the valve holder 500. When the prosthetic valve is to be removed from the valve holder 500, the second portion 532 of each mating feature 530 may be compressed or bent towards the central opening 515 to aid in removing the stabilization arch from the indentation 535.

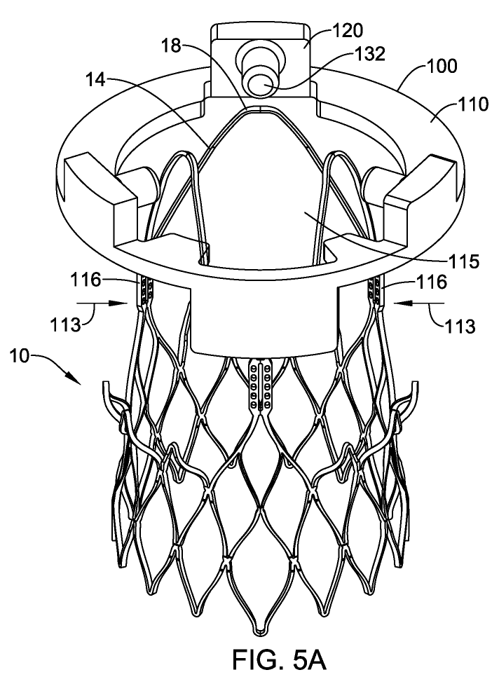

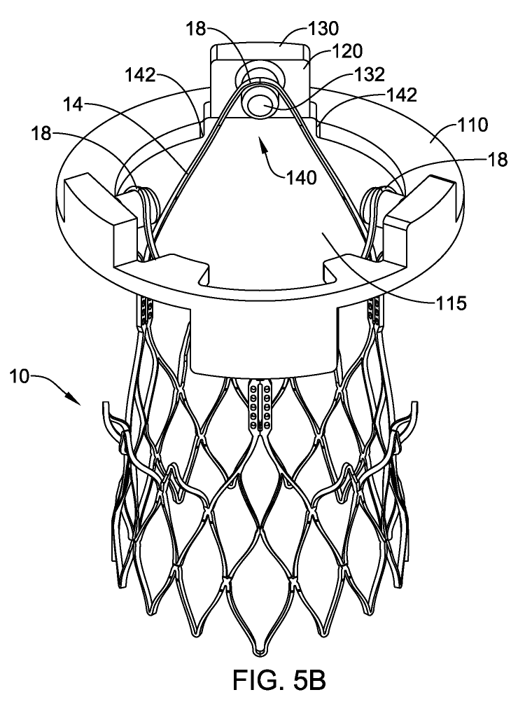

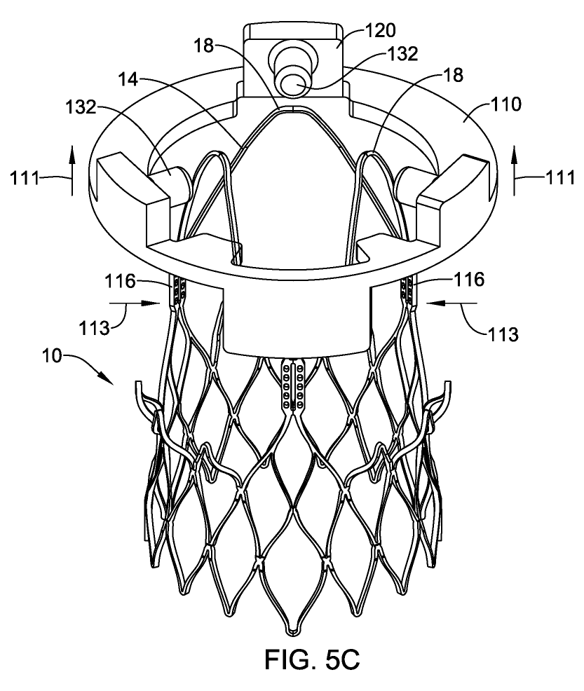

A method of securing an expandable prosthetic heart valve 10 to the valve holder 100 is illustrated in FIGS. 5A-5C. In FIG. 5A the user applies a gentle compressing force, indicated by arrows 113, on commissure posts 116 of the prosthetic heart valve 10 that is in an expanded configuration. Each commissure post 116 joins adjacent stabilization arches 14. In the illustrated embodiment, the prosthetic heart valve 10 has three commissure posts 116, and the user may apply a compressing force onto all three of the commissure posts 116. The user inserts the stabilization arches 14 of the valve into the central opening 115 of the valve holder 100 from the bottom of the valve holder 100 as shown in FIG. 5A. The user aligns the apex 18 of each stabilization arch 14 of the prosthetic heart valve 10 with a corresponding mating feature 120 on the valve holder 100. In this embodiment, the mating features 120 each include a tab 130 extending perpendicularly (i.e., axially upward) from the upper surface of the ring body 110 and a post 132 extending radially inward, so the apex 18 of each stabilization arch 14 may be placed over one post 132. See FIG. 5B. The prosthetic heart valve is then allowed to expand such that each stabilization arch 14 engages with one mating feature 120. The inner diameter of the central opening 115 may be slightly less than the outer diameter of the prosthetic heart valve 10 in the expanded configuration, providing an interference fit between the stabilization arches 14 and the mating features 120. As shown in FIG. 5B, the ring body may include a notch or cut-out portion 140 aligned below each post 132, and the step of aligning the apex 18 of each stabilization arch 14 includes inserting the prosthetic heart valve 10 into the central opening 115 from the lower surface of the ring body and placing the apex 18 of each stabilization arch 14 over one of the posts 132 with opposing sides of the stabilization arch 14 contacting the opposing edges 142 of the notch or cut-out portion 140 below the post 132. This provides three points of contact for each stabilization arch 14 with the ring body 110. FIG. 5C illustrates removal of the prosthetic heart valve 10 from the valve holder 100. The user applies a gentle compressing force, indicated by arrows 113, on the commissure posts 116 on the prosthetic heart valve 10 while grasping the valve holder 100. When the stabilization arches 14 clear the inner surface of each post 132, the valve holder 100 may be moved away from the prosthetic heart valve 10, indicated by arrows 111.

The materials that can be used for the various components of the medical device system and the various elements thereof disclosed herein may include those commonly associated with medical devices. For simplicity purposes, the following discussion refers to the prosthetic heart valve holder.

In some embodiments, the prosthetic heart valve holder and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A prosthetic heart valve holder, comprising:
a ring body defining a central opening; and
a plurality of mating features disposed on the ring body and facing the central opening, each mating feature configured to engage with one of a plurality of stabilization arches of a prosthetic heart valve when the prosthetic heart valve is in an expanded configuration.

2. The prosthetic heart valve holder of claim 1, wherein each mating feature is configured to engage one of the plurality of stabilization arches at at least two contact points.

3. The prosthetic heart valve holder of claim 1, wherein the plurality of mating features includes three mating features positioned at 120-degree intervals.

4. The prosthetic heart valve holder of claim 1, wherein each of the plurality of mating features includes a tab extending axially upwards from a top surface of the ring body, and at least a first tab includes a post extending from the tab radially into the central opening.

5. The prosthetic heart valve holder of claim 4, wherein each of the plurality of mating features includes a cut-out portion of the ring body adjacent a base of the tab, with the post centered above the cut-out portion.

6. The prosthetic heart valve holder of claim 4, wherein each of the tabs include the post extending radially into the central opening.

7. The prosthetic heart valve holder of claim 4, wherein a second tab and a third tab each includes an opening extending radially through the tab, wherein the prosthetic heart valve holder further includes a rod configured to extend across a portion of the central opening and through the openings in the second and third tabs.

8. The prosthetic heart valve holder of claim 1, wherein the ring body includes a handle, wherein at least a portion of the handle is configured to extend axially upwards above an upper surface of the ring body and extending across the central opening.

9. The prosthetic heart valve holder of claim 8, wherein the handle has a first configuration in which it extends laterally across the central opening and does not extend axially above the upper surface of the ring body, and a second configuration in which at least the portion of the handle rotates to extend axially above the upper surface of the ring body.

10. The prosthetic heart valve holder of claim 1, wherein each of the plurality of mating features includes an arm having a first portion extending axially upwards from an upper surface of the ring body, and a second portion extending at an angle from the first portion.

11. A prosthetic heart valve holder, comprising:
a ring body; and
a plurality of engagement members extending axially upward from an upper surface of the ring body, each of the plurality of engagement members configured to engage an apex of one of a plurality of stabilization arches of a prosthetic heart valve when the prosthetic heart valve is in an expanded configuration.

12. The prosthetic heart valve holder of claim 11, wherein each of the plurality of engagement members includes a notch in the ring body, each notch configured to engage opposing sides of a single stabilization arch.

13. The prosthetic heart valve holder of claim 12, wherein the plurality of engagement members each includes a tab extending axially from the ring body, wherein at least one tab has an extension configured to engage the apex of one of the plurality of stabilization arches at a position axially spaced apart from the notch.

14. The prosthetic heart valve holder of claim 11, wherein each of the plurality of engagement members includes a tab extending axially upwards from a top surface of the ring body, and at least a first tab includes a post extending from the tab radially into a central opening of the ring body.

15. The prosthetic heart valve of claim 14, wherein each of the tabs include the post extending from the tab radially into the central opening.

16. The prosthetic heart valve holder of claim 14, wherein a second tab and a third tab each includes an opening extending radially through the tab, wherein the prosthetic heart valve holder further includes a rod configured to extend across a portion of the central opening and through the openings in the second and third tabs, wherein the apex of each stabilization arch extends over the rod.

17. The prosthetic heart valve holder of claim 11, wherein the ring body includes a handle, wherein at least a portion of the handle is configured to extend axially upwards above the upper surface of the ring body and to extend across a central opening of the ring body.

18. A method of securing an expandable prosthetic heart valve to a valve holder comprising:
compressing commissure posts of a prosthetic heart valve in an expanded configuration, wherein each commissure post joins adjacent stabilization arches;
aligning an apex of each one of a plurality of stabilization arches of the prosthetic heart valve with a corresponding mating feature on the valve holder; and
allowing the prosthetic heart valve to expand such that each stabilization arch engages with one mating feature.

19. The method of claim 18, wherein the valve holder includes a ring body defining a central opening and the mating feature includes a plurality of tabs extending axially upward from an upper surface of the ring body, wherein at least a first tab includes a post extending radially into the central opening, wherein aligning the apex of each stabilization arch includes inserting the prosthetic heart valve into the central opening from a lower surface of the ring body and placing the apex of one stabilization arch over the post.

20. The method of claim 19, wherein the ring body includes three tabs each with the post extending radially into the central opening, wherein the ring body further includes a notch aligned below each post, wherein aligning the apex of each stabilization arch includes inserting the prosthetic heart valve into the central opening from the lower surface of the ring body and placing the apex of each stabilization arch over one of the posts and opposing sides of each stabilization arch within the notch.
